(19) United States
(12) Patent Application Publication
(10) Pub. No.: US 2016/0037823 A1
(43) Pub. Date: Feb. 11, 2016

(54) Medical therapy using cigarettes

(71) Applicant: Aari Ruben, Tucson, AZ (US)

(72) Inventor: Aari Ruben, Tucson, AZ (US)

(21) Appl. No.: 14/121,187

(22) Filed: Aug. 11, 2014

(51) Int. Cl. A24D 3/06; A61M 15/06; A61M 15/00

(52) U.S. Cl. CPC A24D 3/061; A61M 15/0001; A61M 15/0031; A61M 15/06; A61M 2205/36

(57) ABSTRACT

The invention is a medical treatment mechanism. Using an organic material which is ignited and inhaled by a patient, such as tobacco or cannabis, a breakable capsule containing the medicine is contained and selectively released so that heat from the ignited organic material activates the medicine. The medicine is released, ideally by the patient breaking the capsule, the medicine is inhaled by the patient for treatment of a medical condition.

### BACKGROUND OF THE INVENTION

This invention relates generally to medical treatments and more particularly to the use of cigarettes, tobacco based products, and marijuana for the application of a medical treatment.

Medical delivery systems have been developing over the past decade with a variety of mechanisms being employed. These include inhalers, vaporizers, and the like which are used to deliver some level of medical treatment to the lungs of the patient.

The key to any sort of treatment and its use by the patient is the simplicity or lack of disruption to the patient's natural routine. The more disruptive the treatment is to the patient's life style, the less likely the treatment will be utilized. The push therefore is to find treatment mechanisms that fit naturally into the patient's lifestyle.

Unfortunately, almost all medical delivery systems require a complete break from the life's activity, thereby making the treatment less likely to be used.

It is clear from the foregoing that there is a need for a medical delivery system which is minimally disruptive to the patient.

### SUMMARY OF THE INVENTION

The present invention uses ignited organic material (such as tobacco or cannabis) to heat a medicine released from a breakable capsule so that the medicine and the smoke from the ignited organic material is inhaled into the lungs of the patient.

Within the discussion below, the preferred delivery mechanism is in the form of a cigarette made from tobacco, cannabis, or some other ignitable organic material. The invention though is not intended to be so limited but includes other delivery systems such as: cigars, cigarette holders, hookahs, and other such mechanisms well known to those of ordinary skill in the art.

The invention utilizes breakable capsules as a dosing mechanism for the medicine. In some embodiments of the invention a single breakable capsule is used while in others, multiple breakable capsules are implanted into the cigarette. In use, the patient/user breaks these capsules using mechanical force which is applied by clamping down with the teeth onto the capsule, breaking the capsule by finger force, or through a “bayonet” type of mechanism which punctures the capsule to release the medicine.

Breakable capsules are well known in the art and include such configurations as, but not limited to those shown in: U.S. Pat. No. 7,984,719, entitled Filtered Cigarette Incorporating a Breakable Capsule” issued to Dube et al. on Jul. 26, 2011; U.S. Pat. No. 8,470,215, entitled “Process for Manufacturing Breakable Capsules Useful in Tobacco Products” issued to Zhang on Jun. 25, 2013; both of which are incorporated hereinto by reference.

Within the breakable capsule is a medicine which ideally is enhanced by the heat from the ignited organic material. In some applications, the medicine does not react to the heat in any measureable manner but rather is used in conjunction with the medical aspect of the organic material itself. This is particularly true when the organic material is cannabis.

Cannabinoids are a class of diverse chemical compounds. Cannabinoids act on cells to repress neurotransmitter release in the brains. These receptors proteins include endocannabinoids which naturally occur in the body, phytocannabinoids found in cannabis and other plants, and synthetic cannabinoids which are man-made. There are at least eighty-five different cannabinoids which may be isolated from cannabis. These cannabis derived cannabinoids have a varied effect on the neurotransmitters and as such have varied treatment potentials.

Those of ordinary skill in the art readily recognize a wide variety of chemicals which have therapeutic affects when inhaled. These include, but are not limited to those described in: U.S. Pat. No. 8,324,122, entitled “Aroma-therapeutic Bedding Set” issued to Juhasz et al. on Dec. 4, 2012; U.S. Pat. No. 8,4565,417, entitled “Personal Care Compositions with Improved Hyposensitivity” issued to Cahen on Jun. 4, 2013; U.S. Pat. No. 8,512,723, entitled “Antimicrobial Compositions and Methods” issued to Scholz, et al. on Aug. 20, 2013; U.S. Pat. No. 8,637,553, entitled “Fluoro Substituted Omega-Carboxyaryl Diphenyl Urea for the Treatment and Prevention of Diseases and Conditions” issued to Boyer et al. on Jan. 28, 2014; U.S. Pat. No. 8,754,039, entitled “Targeted Antimicrobial Moieties” issued to Eckert et al. on Jun. 17, 2014; U.S. Pat. No. 8,776,786, entitled “Pulse Drug Nebulization System, Formulations therefore, and Method of Use” issued to Kraft et al. on Jul. 15, 2014; U.S. Pat. No. 8,778,344, entitled “Method of Treating Atopic Dermatitis with Antibody to Zeytorl7 Ligand” issued to Sprecher, et al. on Jul. 15, 2014; and, U.S. Pat. No. 8,778,394, entitled “Small-Volume Oral Transmucosal Dosage Forms” issued to Palmer et al. on Jul. 15, 2014; all of which are incorporated hereinto by reference.

The medicine is ideally contained within the capsule and then inserted into the cigarette using a variety of techniques.

Those of ordinary skill in the art readily recognize a variety of techniques which might be employed to insert the breakable capsules into the cigarettes, including, but not limited to: U.S. Pat. No. 8,574,141, entitled “Apparatus for Inserting Objects into a Filter Component of a Smoking Article” issued to Barnes et al. on Nov. 5, 2013; incorporated hereinto by reference.

The patient/user ignites the cigarette and breaks the capsule either manually or by biting down on the capsule to release the medicine which is inhaled into the patient's lungs during the natural process of smoking the cigarette.

Ideally the medicine is in oil form so that the heat from the ignited organic material readily vaporizes the medicine for proper inhalation and absorption by the patient. This heated application of the medicine provides for greatly enhanced application of certain medicines.

In the case where the ignited organic material is cannabis, the cannabinoids from the cannabis treat certain conditions of the patient such as glaucoma, pain remediation, nausea, Multiple Sclerosis (MS), and a host of conditions being identified at this time. It has also been shown that cannabis mitigates the effects of smoking on the lungs.

In some situations, the medicine within the capsule is a selected oil from cannabinoids or terpenoids. This oil is used when the ignitable organic material is tobacco, or with cannabis to enhance the treatment being delivered by the ignitable cannabis.

Still another type of material/medicine which is used within the breakable capsules is known as “essential oils”. Essential oils are concentrated hydrophobic liquids containing volatile aroma compounds such as Eucalyptus oil, Rose oil, Lavender and Balsam of Peru.

Ideally, the breakable capsule(s) is/are contained with the filter of the cigarette. Also, ideally the contents of the medicine is imprinted on the cigarette box or wrapping to give the user/patient notice of what medicines are being applied.

Those of ordinary skill in the art readily recognize a variety of filters which may be used in this context, including, but not limited to those described in: U.S. Pat. No. 8,739,802, entitled “Filtered Cigarette” issued to Fagg on Jun. 3, 2014; incorporated hereinto by reference.

While the above discussion has focused on cigarettes containing the breakable capsules with medicine, the present invention is not intended to be so limited. Those of ordinary skill in the art recognize a variety of other such devices, including, but not limited to those described in: U.S. Pat. No. 7,861,726, entitled “Filtration Device for Tobacco Products” issued to Lukasavitz on Jan. 4, 2011; U.S. Pat. No. 8,678,013, entitled “Smoking Article” issued to Crooks et al. on Mar. 25, 2014; and U.S. Pat. No. 8,235,056, entitled “Smoking Article with Concentric Hollow Core in Tobacco Rod and Capsule Containing Flavorant and Aerosol Forming Agents in the Filter System” issued to Zhuang et al. on Aug. 7, 2012; all of which are incorporated hereinto by reference.

The invention, together with various embodiments thereof, will be more fully explained by the accompanying drawings and the following description.

### DRAWINGS IN BRIEF

FIG. 1A illustrates the preferred embodiment of the invention in the form of a cigarette.

FIG. 1B illustrates an alternative embodiment of the invention which utilizes multiple breakable capsules.

FIG. 2 illustrates a cigar embodiment of the invention utilizing a manually operated bayonet to pierce the breakable capsule.

FIG. 3 illustrates an embodiment of the invention deployed in a cigarette holder.

FIG. 4 illustrates an embodiment of the invention deployed in a hookah.

### DRAWINGS IN DETAIL

FIG. 1A illustrates the preferred embodiment of the invention in the form of a cigarette.

This embodiment of a cigarette has two major components, the ignitable organic material 10B and the filter portion 10A. The end of the ignitable organic material 10B is ignited 11 and the user/patient drawn air 12 into the cigarette, through the unignited organic material 10B, through the filter 10A and finally into the user's/patient's lungs 12C.

Breakable capsule 13, in this embodiment, is contained within filter 10A and is selectively crushed/broken when the user/patient applies pressure with their teeth 14A and 14B as illustrated by arrows 15A and 15B. By breaking capsule 13, the medicine contained therein is released into the air stream 12B/12C and ideally is enhanced by the heat within this airstream.

In some situations, the medicine within the capsule is a selected oil from cannabinoids or terpenoids. Other medicines which are containable within breakable capsule 13 have been discussed above.

In one embodiment of the invention, the ignitable organic material 10B is tobacco; in other embodiments the ignitable organic material is cannabis. In either case, an oil from cannabinoids may be used. As noted earlier, cannabinoids act on cells to repress neurotransmitter release in the brain and as such provide a therapeutic treatment on the user/patient.

In the preferred embodiment, band 16 is secured to the cigarette and contains information on the medicine contained with the breakable capsule. In this manner, specific treatments are assured.

FIG. 1B illustrates an alternative embodiment of the invention which utilizes multiple breakable capsules.

In this embodiment, filter 10A has multiple breakable capsules 13A contained therein. Each of these breakable capsules contain medicine(s) adapted to treat a condition of the user/patient. This embodiment is particularly powerful where multiple types of medicines are to be used allowing the manufacturer of the cigarette to formulate a specific treatment for the user/patient.

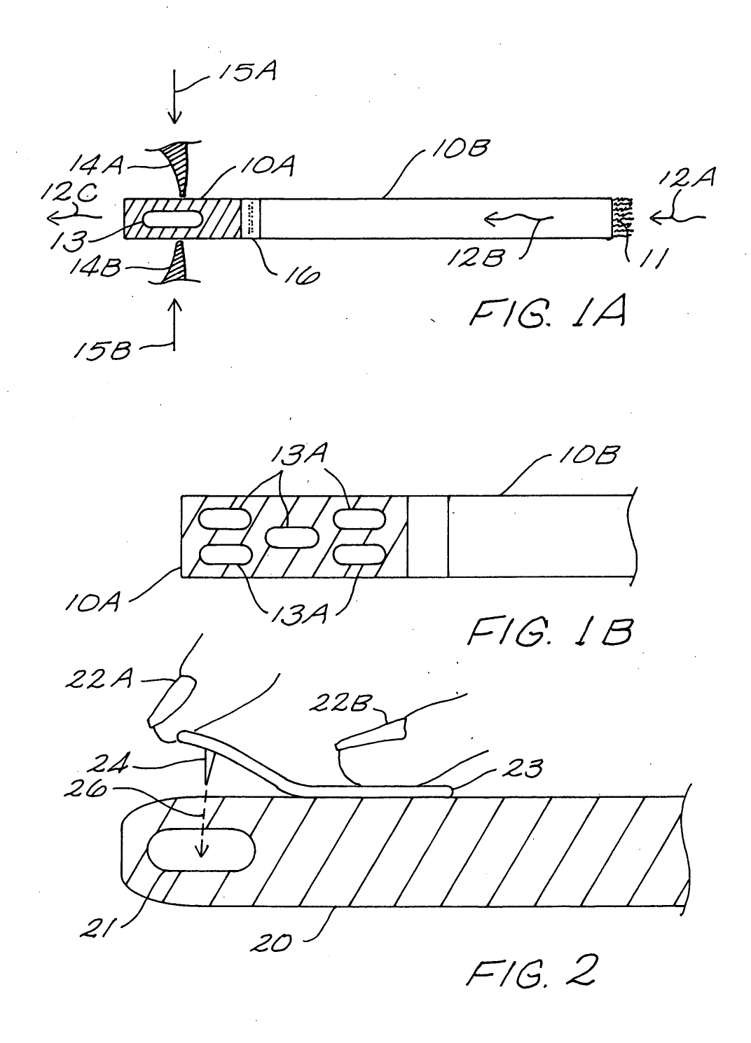

FIG. 2 illustrates a cigar embodiment of the invention utilizing a manually operated bayonet to pierce the breakable capsule.

Cigar 20 has breakable capsule 21 contained therein. To release the medicine within breakable capsule 21, in this embodiment, mechanism 23 is held by the thumb of user/patient 22B while lifting the opposing end of mechanism 23 using finger 22A. When finger 22A releases the end of mechanism 23, spring action pushes bayonet into breakable capsule 21 as indicated by arrow 26.

Those of ordinary skill in the art readily recognize a variety of mechanisms which can be used in this context to manually break capsule 21 to release the enclosed medicine therein. Cigar 20 is ignited and inhaled by the patient/user.

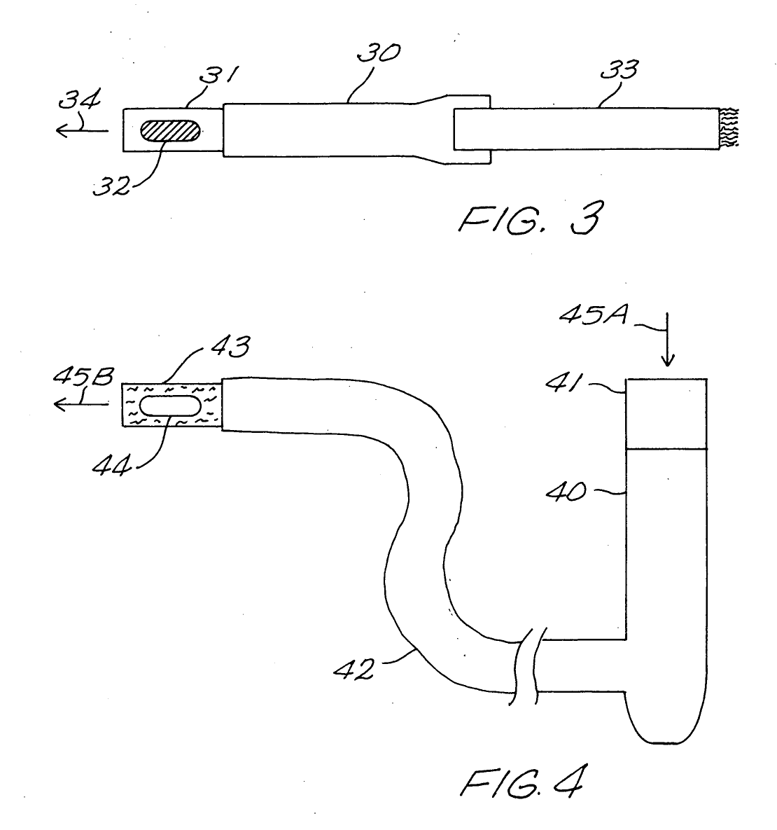

FIG. 3 illustrates an embodiment of the invention deployed in a cigarette holder.

Cigarette holder 30 is adapted to hold cigarette 33, allowing the user/patient to draw air through cigarette 33 and cigarette holder 30 as indicated by arrow 34.

Cigarette holder 30 is also adapted to receive medicated end 31 which contains the breakable capsule 32 having the medicine therein. When the breakable capsule 32 is broken, the medicine contained therein is communicated to the patient/user within air stream 34.

In this embodiment, medicated end 31 is replaceable so that once used, it is removed from the by cigarette holder 30 and discarded. A new medicated end 31 is used for later treatment.

FIG. 4 illustrates an embodiment of the invention deployed in a hookah.

Hookah 40 burns organic material contained within top 41. Air is drawn through the hookah 40, piping 42, and then through medicated end 43 as indicated by arrows 45A and 45B.

As before, medicated end 43 is removable from the assembly and contains a breakable capsule 44 containing the medicine which is to be applied to the patient/user.

Because of the removable nature of the medicated end 43, the same hookah 40 assembly can be used to deliver a variety of medicines.

It is clear that the present invention provides a medical delivery system that fits into the life style for some patients.

What is claimed is:

1. A medical treatment apparatus comprising:
a) a quantity of organic material adapted to be ignited and inhaled by a patient; and,
b) at least one breakable capsule interposed between the ignited organic material and a patient's lungs, said breakable capsule containing a quantity of medicine, said medicine being enhanced by heat from the ignited organic material.

2. The medical treatment apparatus according to claim 1, wherein said at least one breakable capsule is adapted to be broken by a user to release the medicine.

3. The medical treatment apparatus according to claim 2, wherein said medicine is in oil form.

4. The medical treatment apparatus according to claim 3, wherein said medicine includes oil from cannabinoids.

5. The medical treatment apparatus according to claim 3, wherein said medicine includes Eucalyptus oil.

6. The medical treatment apparatus according to claim 3, wherein said medicine includes essential oils.

7. The medical treatment apparatus according to claim 3, wherein said medicine includes rose oils.

8. The medical treatment apparatus according to claim 3, wherein said organic material is contained within a cigarette and wherein said at least one breakable capsule is contained within a cigarette filter attached thereto.

9. The medical treatment apparatus according to claim 8, wherein said organic material includes a quantity of marijuana.

10. The medical treatment apparatus according to claim 8, wherein said organic material includes a quantity of tobacco.

11. The medical treatment apparatus according to claim 3, wherein said tobacco and said at least one breakable capsule is contained within a cigar.

12. The medical treatment apparatus according to claim 3,
a) further including a cigarette holder;
b) wherein said tobacco is contained within a cigarette held by said cigarette holder; and,
c) wherein said at least one breakable capsule is attached to said cigarette holder.

13. A medical treatment apparatus comprising:
a) a cigarette made from tobacco and having a filter thereon, said cigarette adapted to be ignited and inhaled by a patient; and,
b) at least one breakable capsule contained within said filter, said at least one breakable capsule containing a quantity of medicine, said medicine being enhanced by heat from the ignited tobacco.

14. The medical treatment apparatus according to claim 13, wherein said at least one breakable capsule is adapted to be broken by a user to release the medicine.

15. The medical treatment apparatus according to claim 14, wherein said medicine includes oil from cannabinoids.

16. The medical treatment apparatus according to claim 15, wherein said at least one breakable capsule is at least two breakable capsules, and wherein one of said at least two breakable capsules includes a second medicine adapted to enhance a medical treatment associated with the oil from cannabinoids.

17. A medical treatment apparatus comprising:
a) a cigarette made from cannabis, said cigarette adapted to be ignited and inhaled by a patient; and,
b) at least one breakable capsule contained within said cigarette, said at least one breakable capsule containing a quantity of medicine, said medicine being enhanced by heat from the ignited cannabis.

18. The medical treatment apparatus according to claim 17, wherein said at least one breakable capsule contains a medicine adapted to enhance the cannabis.

19. The medical treatment apparatus according to claim 18, wherein said cigarette further includes a filter containing said at least one breakable capsule.
